(19) United States
(12) Plant Patent Application Publication
(10) Pub. No.: US 2008/0141407 P1
(43) Pub. Date: Jun. 12, 2008

(54) Loblolly pine tree named 'CF L3791'

(76) Inventors: John Pait, Atlanta, GA (US); Plamen Denchev, Victoria (CA); Stephen Attree, Victoria (CA); Robert J. Weir, Cary, NC (US); Andy Benowicz, Victoria (CA)

Correspondence Address: MICHAEL BEST & FRIEDRICH LLP, Two Prudential Plaza, 180 North Stetson Avenue, Suite 2000, CHICAGO, IL, 60601, US

(21) Appl. No.: 11/635,703

(22) Filed: Dec. 7, 2006

(51) Int. Cl. A01H 7/00

(52) U.S. Cl. PLT/213

(57) ABSTRACT

A new and distinctive variety of a loblolly pine tree which has been denominated varietally as ‘CF L3791’ which is distinguished by high resistance to fusiform rust and pitch canker, excellent stem straightness, medium to wide crown width, few whorls to medium number of whorls, and very fast growth.

### LATIN NAME

Pinus taeda

### VARIETY DENOMINATION

CF L3791

### BACKGROUND

A new variety of loblolly pine tree (Pinus taeda), has been discovered. This selection has been designated as CF L3791.

This new variety is a progeny of two first generation selections. Female parent is a first generation selection made in Williamsburg County, South Carolina. Male parent is a first generation selection made in Onslow County, North Carolina.

Cross pollination occurred in early 1997 followed by induction and cryopreservation of embryogenic tissue in 1998. First somatic seedlings were produced in 2000 and planted in early 2001 in seven field experiments. A total of 61 ramets were planted ranging from 5 to 11 ramets per field experiment. The field experiments are located in Mississippi, Florida, Georgia and South Carolina.

### BRIEF SUMMARY

A new and distinct cultivar of loblolly pine (Pinus taeda) is distinctly characterized by great resistance to fusiform rust and pitch canker, high growth rate, excellent stem straightness, medium to wide crown width, few whorls to medium number of whorls, and which is mature for commercial harvesting sooner than conventionally grown trees under the ecological conditions prevailing in the Piedmont, Atlantic and Gulf Coastal Plains, and Mid-Continent regions of the United States.

The Pinus taeda plants of this variety were asexually propagated using an advanced form of micropopagation called somatic embryogenesis carried out at CellFor's production facility in Victoria, Canada. Somatic embryogenesis uses a complex process which relies on the splitting of one embryo into many identical embryos. Somatic embryos can then be grown into plants which are all identical genetically. The asexual propagation occurs at an earlier stage in the plant's life cycle than most other micropropagated plants. The detailed methods for somatic embryogenesis used for asexually propagating conifers in general are described in U.S. Pat. No. 6,372,496 and for loblolly pine in particular in US Patent Application 2004/0203150.

### BRIEF DESCRIPTION OF THE DRAWINGS

The drawings are color photographs showing the new variety of loblolly pine.

FIG. 1 is a photograph showing CF L3791 ramet # 7 planted in Holly Hill, S.C. The picture was taken after five field growing seasons. The picture shows excellent stem straightness, few whorls to medium number of whorls per unit stem length, medium angle between the stem and the branches, and medium to large branch diameter (relative to the size of the stem).

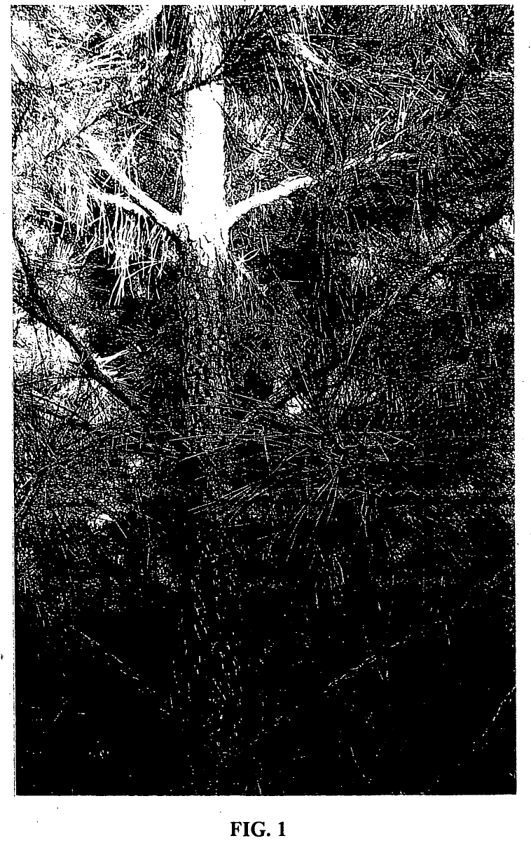

FIG. 2 is a photograph showing CF L3791 ramet # 4 planted in Winokur, Ga. The picture was taken after five field growing seasons. The picture shows superiority of growth and medium to wide crown width.

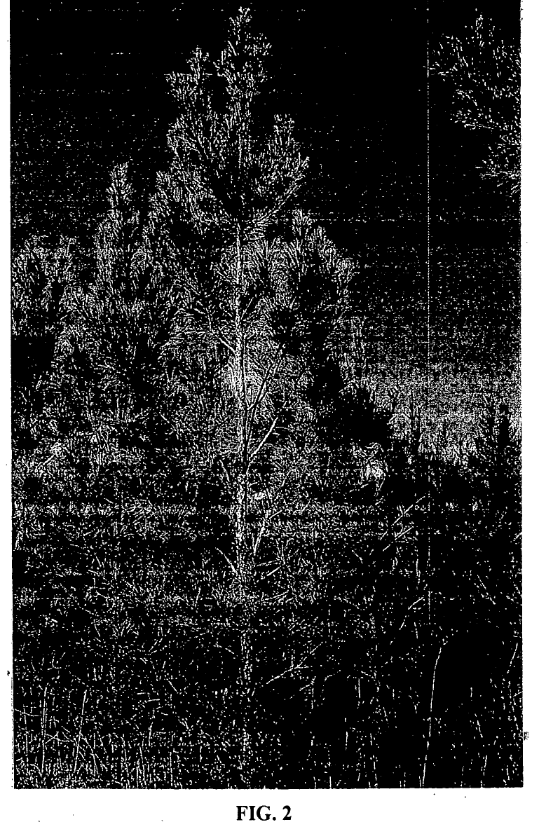

### DETAILED BOTANICAL DESCRIPTION

The botanical details of this new and distinct variety of loblolly pine tree follow.
Leaf: Evergreen needles, 6 to 9 inches long, with (usually) three yellow-green needles per fascicle.
Flower: Monoecious; males long cylindrical, red to yellow, in clusters at branch tips; females yellow to purple.
Fruit: Ovoid to cylindrical, 3 to 6 inch red-brown cones; umbo is armed with a short spine, maturing in early fall.
Twig: Orange-brown in color, fine to moderately stout; buds are narrowly ovoid, light reddish brown.
Bark: Initially red- to gray-brown and scaly; older trees are ridged and furrowed, with somewhat rounded scaly plates; very old trees have red-brown, flat scaly plates.
Form: A medium to large tree can reach well over 100 feet tall, self-prunes well and develops a fairly straight trunk and an oval, somewhat open crown. Compared to unimproved loblolly pine trees, ‘CF L3791’ is characterized by very high growth rate, great resistance to fusiform rust (caused by Cronartium quercuum (Berk.) Miyabe ex Shirai f. sp. fusiforme (Cumm.) Burds. et Snow), great resistance to pitch canker (caused by Fusarium circinatum Nirenburg et O'Donnell), excellent stem straightness, medium to wide crown width, and few whorls to medium number of whorls.
Average height: 26 ft after 5 field growing seasons
Maximum height: 28 ft after 5 field growing seasons
Average diameter at breast height: 4.6 inches after 5 field growing seasons
Maximum diameter at breast height: 6.1 inches after 5 field growing seasons
Percent stem fusiform rust infection at age 5: 2
Percent branch fusiform rust infection at age 5: 6
Percent branch and stem fusiform rust infection at age 5: 0
Percent dead ramets due to fusiform rust infection at age 5: 0
Percent stem fusiform rust infection in the USDA Resistance Screening Center (Asheville, N.C.) tests after artificial inoculation with rust spores: 6% (compared to 78% infection in unimproved seedlings)
Percent of seedlings with over 50% of tissue infected by pitch canker in the USDA Resistance Screening Center (Asheville, N.C.) tests after artificial inoculation with pitch canker spores: 0% (compared to 99% infection in unimproved seedlings)
Percent stem straightness gain over unimproved trees: 18
Branch angle: medium
Branch diameter: medium to large
Number of major whorls: few to medium
Live crown width: medium to wide
Propagation: propagated by somatic embryogenesis
Use: high yield industrial plantations

Although the new variety of loblolly pine tree possesses the detailed characteristics noted above as a result of the growing conditions prevailing in the seven test locations, it is to be understood that the variations of the usual magnitude and characteristics incident to changes in growing conditions, irrigation, fertilization, pruning, pest control, climatic variations and the like are to be expected. An example of ‘CF L3791’ can be found at Plum Creek Oliver year 2001 line trial, Screven county, GA.

Having thus described and illustrated our new variety of loblolly pine tree, what we claim as new and desire to secure by Plant Letter Patent is:

1. A new and distinct variety of loblolly pine tree named CF L3791 substantially as described and illustrated.
